(19) United States
(12) Patent Application Publication
(10) Pub. No.: US 2026/0207363 A1
(43) Pub. Date: Jul. 23, 2026

(54) CATHETER AND METHOD FOR MANUFACTURING CATHETER

(71) Applicant: TERUMO KABUSHIKI KAISHA, Tokyo (JP)

(72) Inventors: Hiroki YOSHIDA, Odawara Kanagawa (JP); Shun SASAKI, Fuji Shizuoka (JP); Takehiro SUGIMOTO, Fujinomiya Shizuoka (JP)

(21) Appl. No.: 19/570,049

(22) Filed: Mar. 17, 2026

Related U.S. Application Data
(63) Continuation of application No. PCT/JP2024/025927, filed on Jul. 19, 2024.

(30) Foreign Application Priority Data
Sep. 26, 2023 (JP) 2023-163653

(51) Int. Cl. A61F 2/966; A61F 2/962; A61M 25/00; A61M 25/06; A61M 25/09

(52) U.S. Cl. CPC A61F 2/966; A61F 2002/9623; A61M 25/0009; A61M 2025/0681; A61M 25/09

(57) ABSTRACT

A catheter includes a tube, a sheath surrounding the tube and including: a tube body formed of resin, and a reinforcing body having a repeating structure formed of metal strands, and a traction wire connected to a proximal portion of the sheath and configured to move the sheath with respect to the tube. The tube body and the reinforcing body extend continuously over an entire length of the sheath.

### CROSS-REFERENCE TO RELATED APPLICATION

This application is a continuation of International Patent Application No. PCT/JP2024/025927 filed Jul. 19, 2024, which is based upon and claims the benefit of priority from Japanese Patent Application No. 2023-163653, filed Sep. 26, 2023, the entire contents of which are incorporated herein by reference.

### TECHNICAL FIELD

The present disclosure relates to a catheter that is inserted into a body lumen for treatment, and a method of manufacturing a catheter.

### BACKGROUND ART

As a method for treating lesions such as stenosis or occlusion that occur in blood vessels, there is intravascular treatment in which treatment is performed from inside the blood vessel using a device percutaneously inserted into the blood vessel. In intravascular treatment, a catheter is used to deliver a diagnostic contrast medium, a guide wire, or a treatment portion to the lesion site.

For example, in the treatment of myocardial infarction or angina pectoris, a method is performed in which a stent is placed at a lesion site (stenotic portion) of the coronary artery using a catheter to secure a space within the coronary artery, and a similar method may also be performed for improving stenotic portions that occur in other blood vessels, bile ducts, trachea, esophagus, urethra, or other body lumens. A stent is classified into a balloon-expandable stent and a self-expanding stent according to function and placement method.

A balloon-expandable stent does not have an expansion function by itself and, after reaching the target site, is expanded by a balloon and fixed in close contact with the lumen. In contrast, a self-expanding stent has an expansion function by itself and is accommodated in a radially compressed state in advance within a catheter. After reaching the target site, the self-expanding stent is released from the catheter, expands by its own expansion force, and is fixed in close contact with the lumen.

There is known a rapid exchange type (RX type) catheter that delivers a self-expanding stent to a target site in a lumen and fixes the self-expanding stent in close contact with the lumen. The catheter of Patent Literature 1 is formed such that the outer diameter of a distal portion that accommodates the stent is larger than the outer diameter of a proximal portion, and has a tapered portion that increases in diameter toward the distal side between the distal portion and the proximal portion.

### SUMMARY

Such a conventional catheter is provided with a mesh-like or net-like reinforcing body at the distal portion that accommodates the stent. The catheter is also provided with a reinforcing body at the proximal portion. The distal portion in which the stent is accommodated has a large inclination angle of the strands forming the reinforcing body with respect to the longitudinal axis direction, and has high tensile strength in the radial direction. On the other hand, the proximal portion has a small inclination angle of the strands forming the reinforcing body with respect to the longitudinal axis direction, and has high tensile strength in the longitudinal axis direction.

The conventional catheter is not provided with a reinforcing body at the part of the tapered portion formed between the distal portion and the proximal portion. Therefore, a local decrease in tensile strength occurs at the part where the reinforcing body is not provided. As a result, the conventional catheter may undergo breakage at the part where the reinforcing body is not provided when a tensile force is applied to the catheter, such as during deployment of the stent.

Embodiments of the present disclosure provide a catheter and a method for manufacturing a catheter that have continuous tensile strength from the distal end to the proximal end and do not cause a local decrease in tensile strength.

In one embodiment, a catheter comprises: a tube; a sheath surrounding the tube and including: a tube body formed of resin, and a reinforcing body having a repeating structure formed of metal strands; and a traction wire connected to a proximal portion of the sheath and configured to move the sheath with respect to the tube. The tube body and the reinforcing body extend continuously over an entire length of the sheath.

The catheter configured as described above has the reinforcing body extending over the entire length of the sheath that is pulled by the traction wire, and thus the sheath has continuous tensile strength in the long axis direction from the distal end to the proximal end, and a local decrease in tensile strength can be prevented from occurring.

### BRIEF DESCRIPTION OF DRAWINGS

FIG. 1 is a front view of a catheter according to an embodiment.

FIG. 2A is a cross-sectional view illustrating a distal side of the catheter before deployment of the stent.

FIG. 2B is a cross-sectional view illustrating the distal side of the catheter during deployment of the stent.

FIG. 3 is an enlarged cross-sectional view in the vicinity of a boundary between a first region and a second region of a first sheath.

FIG. 4 is a partial front view of a first sheath reinforcing body in the first region.

FIG. 5 is a partial front view of a first sheath reinforcing body in the second region.

FIG. 6 is an enlarged cross-sectional view in the vicinity of a boundary between a third region and a fourth region of a second sheath.

FIG. 7A is a cross-sectional view illustrating a manufacturing process of the first sheath in which a core material is inserted into a tube.

FIG. 7B is a cross-sectional view illustrating the manufacturing process of the first sheath in the middle of inserting the tube into a mold.

FIG. 8 is a cross-sectional view illustrating the distal side of the catheter according to a first modified example.

FIG. 9 is a cross-sectional view illustrating the distal side of the catheter according to a second modified example.

FIG. 10 is an enlarged cross-sectional view in the vicinity of the boundary between the first region and the second region, and in the vicinity of the boundary between the second region and a fifth region, in the sheath included in the catheter of FIG. 9.

FIG. 11 is a cross-sectional view of a sheath having a tapered portion according to a modified example.

### DETAILED DESCRIPTION

Hereinafter, embodiments of the present disclosure will be described with reference to the drawings. Note that, the dimensions in the drawings may be exaggerated or may differ from actual dimensions for convenience of description in some cases. In the present specification and the drawings, components having substantially the same functional configuration will be given the same reference numerals, and the redundant description thereof will not be repeated. In the present specification, a side to be inserted into a lumen is referred to as a “distal side” and a side to be operated by a user is referred to as a “proximal side”.

A catheter 10 according to an embodiment is for placing a stent 20, which is a medical deployment object, at a stenosed part, occluded part, or the like occurring in a body lumen such as a blood vessel, bile duct, trachea, esophagus, urethra, or other body lumen, to maintain the patency of the body lumen. The catheter 10 is a rapid exchange type (RX type) in which a guide wire 200 is inserted only into the distal side.

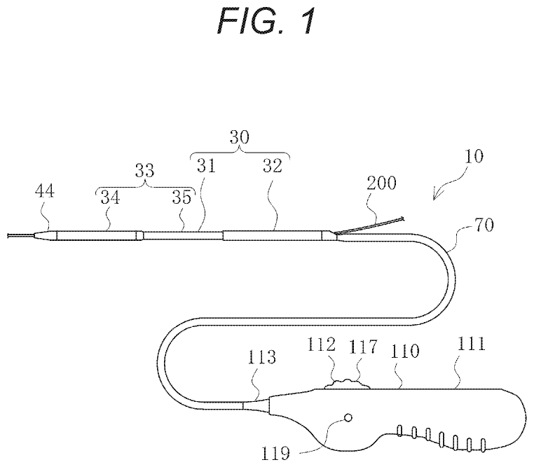

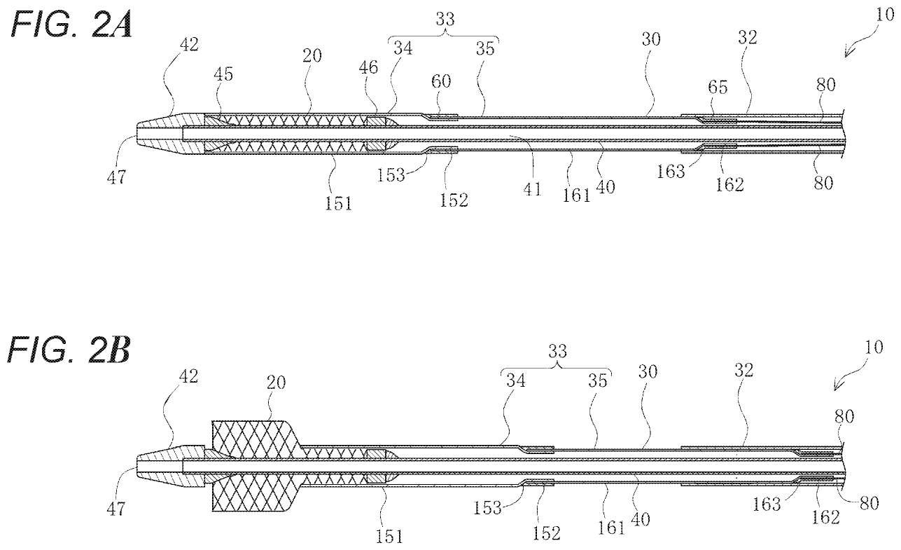

As illustrated in FIGS. 1, 2A, and 2B, the catheter 10 includes the stent 20, an outer tube 30 accommodating the stent 20, an inner tube 40 disposed inside the outer tube 30, and a proximal shaft 70 disposed on the proximal side of the outer tube 30. The catheter 10 further includes a traction wire 80 for pulling the outer tube 30 and an operation portion 110 disposed at a proximal portion of the catheter 10.

The outer tube 30 includes a sheath 33 accommodating the stent 20 and a proximal outer tube 32 positioned on the proximal side of the sheath 33. The inner tube 40 is disposed inside the sheath 33 and the proximal outer tube 32. The sheath 33 is movable toward the proximal side with respect to the proximal outer tube 32. The sheath 33 includes a first sheath 34 and a second sheath 35. The stent 20 having a reduced diameter is disposed inside the first sheath 34.

The first sheath 34 includes a first region 151 on the distal side and a second region 152 on the proximal side. The second region 152 has an outer diameter and an inner diameter smaller than those of the first region 151.

The second sheath 35 has an outer diameter and an inner diameter smaller than those of the first region 151 of the first sheath 34. The distal portion of the second sheath 35 is positioned on the outer surface of the second region 152 of the first sheath 34, and is covered with a connection tube 60, and the connection tube 60 is fused and fixed by heating or the like, whereby the first sheath 34 and the second sheath 35 are connected. Note that the connection tube 60 may be fixed with an adhesive or the like.

The second sheath 35 includes a third region 161 on the distal side and a fourth region 162 on the proximal side. The fourth region 162 has an outer diameter and an inner diameter smaller than those of the third region 161.

The third region 161 of the second sheath 35 has an outer diameter smaller than the inner diameter of the proximal outer tube 32, and a part on the proximal side is positioned inside the proximal outer tube 32. When the sheath 33 moves toward the proximal side with respect to the proximal outer tube 32, the second sheath 35 can slide on the inner surface of the proximal outer tube 32 and enter the inside of the proximal outer tube 32.

The traction wire 80 is fixed to the fourth region 162 of the second sheath 35, which constitutes the proximal portion of the sheath 33. The traction wire 80 is positioned on the outer surface of the fourth region 162 which is the proximal portion of the second sheath 35, and is fixed to the second sheath 35 by being covered with a wire fixing tube 65 and fixed with an adhesive or the like. Note that the traction wire 80 may be fixed by being covered with the wire fixing tube 65 and thermally fused by heating or the like.

The proximal outer tube 32 is a tube body capable of internally receiving the second sheath 35 moving toward the proximal side.

The outer diameter of the outer tube 30 is not particularly limited, but is, for example, 0.5 to 10.0 mm, and preferably 1.0 to 5.0 mm. The inner diameter of the outer tube 30 is not particularly limited, but is, for example, 0.4 to 9.0 mm, and preferably 0.8 to 4.5 mm. The length of the outer tube 30 in the axial direction is not particularly limited, and is, for example, 10 to 250 mm, and preferably 20 to 100 mm.

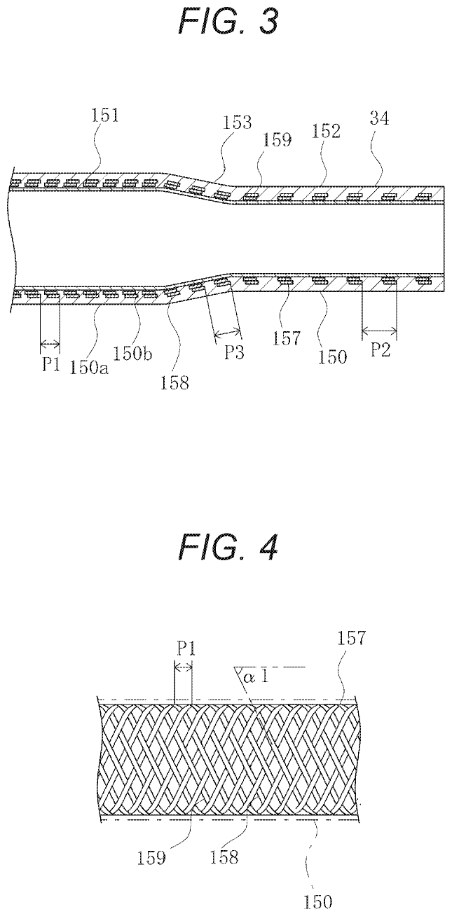

As illustrated in FIG. 3, the first sheath 34 includes a first sheath tube body 150 formed of resin and a first sheath reinforcing body 157 having a repeating structure 159 formed of metal strands 158. The first sheath tube body 150 has an outer layer 150a formed of resin having flexibility and an inner layer 150b formed of resin having low friction properties. The first sheath reinforcing body 157 is positioned between the outer layer 150a and the inner layer 150b. The strands 158 can be formed of stainless steel, gold, platinum, silver, iridium, tungsten, tantalum, or an alloy thereof.

In the first sheath 34, the outer diameter of the first region 151 is larger than the outer diameter of the second region 152, and a part between the first region 151 and the second region 152 is a first tapered portion 153 inclined such that the outer diameter decreases from the distal side toward the proximal side.

The first sheath reinforcing body 157 extends continuously over the entire length from the distal end to the proximal end of the first sheath 34. That is, in the first sheath reinforcing body 157, the repeating structure 159 formed by the continuous strands 158 extends over the first region 151, the first tapered portion 153, and the second region 152.

As illustrated in FIG. 4, the first sheath reinforcing body 157 in the first region 151 has the repeating structure 159 braided in a mesh shape such that the strands 158 intersect each other. The pitch of the repeating structure 159 in the first region 151 is P1, and the inclination angle of the strands 158 with respect to the longitudinal axis direction is α1.

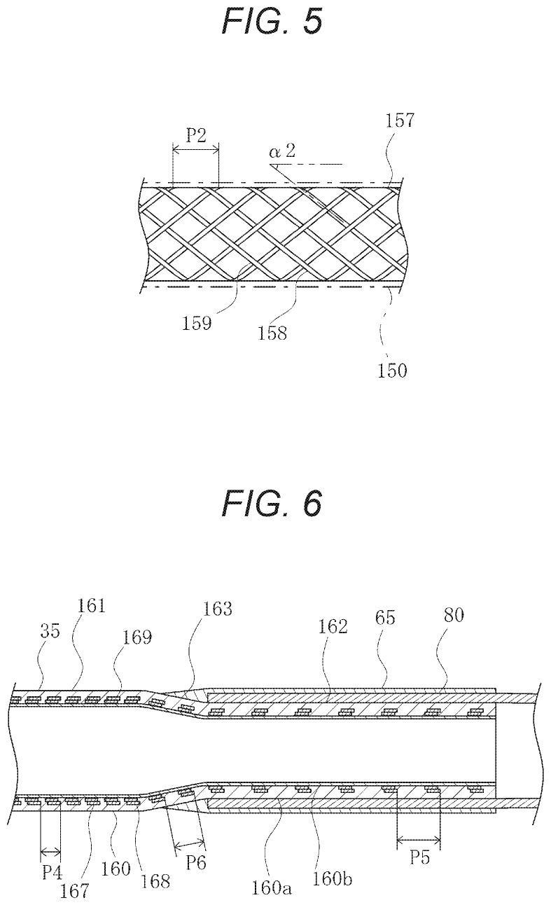

As illustrated in FIG. 5, the first sheath reinforcing body 157 in the second region 152 has the same repeating structure 159 as the first region 157, the pitch thereof is P2, and the inclination angle of the strand 158 with respect to the longitudinal axis direction is α2. The pitch P2 of the repeating structure 159 in the second region 152 is larger than the pitch P1 of the repeating structure 159 in the first region 151. In addition, the inclination angle α2 of the strand 158 in the second region 152 is smaller than the inclination angle α1 of the strand 158 in the first region 151.

The pitch P3 of the repeating structure 159 in the first tapered portion 153 is larger than the pitch P1 in the first region 151 and smaller than the pitch P2 in the second region 152.

As illustrated in FIG. 6, the second sheath 35 includes a second sheath tube body 160 formed of resin and a second sheath reinforcing body 167 having a repeating structure 169 formed of metal strands 168. The second sheath tube body 160 has an outer layer 160a formed of resin having flexibility and an inner layer 160b formed of resin having low friction properties. The second sheath reinforcing body 167 is positioned between the outer layer 160a and the inner layer 160b.

In the second sheath 35, the outer diameter of the third region 161 is larger than the outer diameter of the fourth region 162, and a part between the third region 161 and the fourth region 162 is a second tapered portion 163 inclined such that the outer diameter decreases from the distal side toward the proximal side.

The second sheath reinforcing body 167 extends continuously over the entire length from the distal end to the proximal end of the second sheath 35. That is, the second sheath reinforcing body 167 extends continuously over the third region 161, the second tapered portion 163, and the fourth region 162.

The repeating structure 169 of the second sheath reinforcing body 167 is a mesh-like braided structure similar to the repeating structure 159 of the first sheath reinforcing body 157. The pitch P5 of the repeating structure 169 in the fourth region is larger than the pitch P4 of the repeating structure 169 in the third region. In addition, the pitch P6 of the repeating structure 169 in the second tapered portion 163 is larger than the pitch P4 in the third region 161 and smaller than the pitch P5 in the fourth region 162.

The traction wire 80 fixed to the proximal portion of the second sheath 35 is fixed within the fourth region 162 serving as a fixing region. Since the second sheath reinforcing body 167 extends in the fourth region 162, the traction wire 80 is fixed to the fixing region where the second sheath reinforcing body 167 extends. As described above, since the traction wire 80 is fixed to the fixing region where the second sheath reinforcing body 167 extends, the strength is higher than that in a case where the traction wire is fixed only to a region formed of a flexible resin, and thus the second sheath 35 is less likely to break when the traction wire 80 is pulled.

The constituent material of the outer layers of the sheath 33 and the proximal outer tube 32 is not particularly limited, but is preferably a material having both hardness and flexibility, and for example, nylon, polyester, fluororesin, or the like can be suitably used. The constituent material of the inner layer is preferably a fluororesin. Examples of fluororesins include polytetrafluoroethylene (PTFE), tetrafluoroethylene-perfluoroalkyl vinyl ether copolymer (PFA), and tetrafluoroethylene-hexafluoropropylene copolymer (FEP).

As illustrated in FIG. 2, the inner tube 40 is a tube body through which a guide wire lumen 41 is formed on the distal side of the catheter 10. The inner tube 40 includes a distal tip 44 fixed to a distal portion, a distal marker 45, and a proximal marker 46. The inner tube 40 and the distal tip 44 are aligned in the axial direction to form the common guide wire lumen 41.

The inner tube 40 is at least partially positioned inside the outer tube 30 and extends through the inside of the stent 20 disposed inside the outer tube 30. The distal end of the inner tube 40 is positioned on the distal side of the outer tube 30.

The outer diameter of the inner tube 40 is not particularly limited, but is, for example, 0.15 to 3.0 mm, and preferably 0.3 to 1.5 mm. The inner diameter of the inner tube 40 is not particularly limited, but is, for example, 0.1 to 2.0 mm, and preferably 0.2 to 1.0 mm. The length of the inner tube 40 in the axial direction is not particularly limited, and is, for example, 50 to 2,000 mm, and preferably 100 to 1,000 mm.

The constituent material of the inner tube 40 is not particularly limited, but for example, polyether ether ketone (PEEK), nylon, polyimide, or the like can be suitably used.

The distal tip 44 constitutes a distal portion of the inner tube 40. The guide wire lumen 41 opens at a distal opening 47 formed at the distal end of the distal tip 44. Note that the distal tip 44 may be formed integrally with the inner tube 40. The proximal surface of the distal tip 44 is in contact with or close to the distal surface of the first sheath 34 accommodating the stent 20.

As the constituent material of the distal tip 44, a flexible material is preferably used, and for example, a nylon elastomer, a polyester elastomer, a urethane elastomer, or the like can be suitably used. The constituent material of the distal tip 44 may include a contrast medium.

The traction wire 80 is a wire for pulling the sheath 33 toward the proximal side to release the stent 20 from the sheath 33. One or more (in the present embodiment, two) traction wires 80 are provided. A proximal side of the traction wire 80 is fixed to an actuating portion 112 of the operation portion 110 to be described later. The distal side of the traction wire 80 is fixed to the proximal portion of the second sheath 35.

As the traction wire 80, a wire or a wire obtained by twisting a plurality of wires can be suitably used. The thickness of the traction wire 80 is not particularly limited, but is, for example, 0.05 to 1.0 mm, and preferably 0.1 to 0.5 mm. The surface of the traction wire 80 is coated with a fluororesin which is a low friction material.

The constituent material of the traction wire 80 is not particularly limited, but, for example, wires formed of various metals such as stainless steel wire (preferably, high-tensile stainless steel for springs), piano wire (preferably, piano wire subjected to nickel plating or chrome plating), or superelastic alloy wire, Ni—Ti alloy, Cu—Zn alloy, Ni—Al alloy, tungsten, tungsten alloy, titanium, titanium alloy, cobalt alloy, or tantalum, and relatively high-rigidity polymer materials such as polyamide, polyimide, ultrahigh molecular weight polyethylene, polypropylene, or fluororesin, or a suitable combination thereof, can be preferably applied.

The stent 20 is formed in a substantially cylindrical shape and accommodated in the first sheath 34 in a state of being radially compressed as illustrated in FIG. 2. The stent 20 is pushed out from the opening on the distal side of the first sheath 34, and accordingly, the stress load is removed and the stent expands and returns to the shape before compression. The stent 20 is not limited in form as long as the stent is a so-called self-expanding stent. As an example, the stent 20 may have one substantially cylindrical shape by connecting a plurality of annular portions that are bent into an annular shape.

As the constituent material of the stent 20, a superelastic alloy is suitably used. The superelastic alloy herein is generally called a shape memory alloy, and exhibits superelasticity at least at a body temperature (around 37° C.). As the shape memory alloy, a Ni—Ti alloy, a Cu—Al—Ni alloy, a Cu—Zn—Al alloy, or the like can be suitably used.

As illustrated in FIG. 1, the operation portion 110 includes an operation main body portion 111, an actuating portion 112, and a kink-resistant protector 113.

The operation main body portion 111 is a part gripped by the operator. The operation main body portion 111 accommodates a mechanism including a part of the actuating portion 112 for pulling the traction wire 80.

The actuating portion 112 is a part where the user performs the traction operation of the traction wire 80. The actuating portion 112 includes a dial 117 operated by the operator, and can rotate with respect to the operation main body portion 111 about a rotation shaft 119.

The dial 117 is a part that the operator rotationally operates, is formed in a disk shape, and is disposed in the operation main body portion 111 to be partially exposed from the operation main body portion 111. A part of the dial 117 exposed from the operation main body portion 111 is a part operated by the operator. A surface part that may be touched by the operator when operating the dial 117 is preferably a non-slip surface. For example, the outer peripheral surface of the dial 117 is preferably subjected to knurling treatment, embossing treatment, high friction material coating, and the like. When the operator rotates the dial 117, a winding shaft (not illustrated) provided inside the operation main body portion 111 rotates, and the traction wire 80 can be wound.

The kink-resistant protector 113 partially protrudes from the operation main body portion 111 toward the distal side while covering the proximal side of the proximal shaft 70. The kink-resistant protector 113 suppresses kinking on the proximal side of the proximal shaft 70.

Next, a method for manufacturing the sheath 33 constituting the catheter 10 will be described. First, a tube 300 including a reinforcing body 312 having a repeating structure 313 formed of metal strands 314 is formed over the entire length of a resin tube body 310 having a distal end and a proximal end. The tube 300 has the same outer diameter and inner diameter over the entire length, and the pitch of the repeating structure 313 of the reinforcing body 312 is also formed to be the same over the entire length.

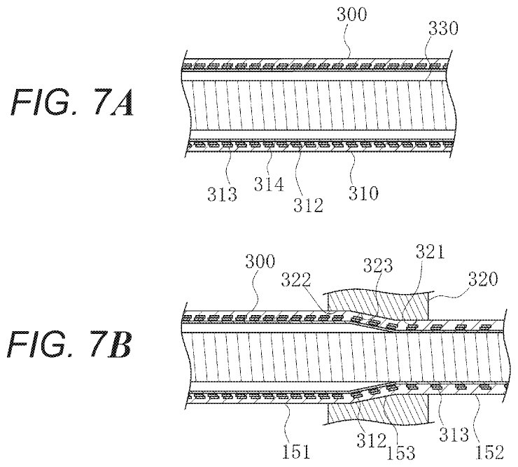

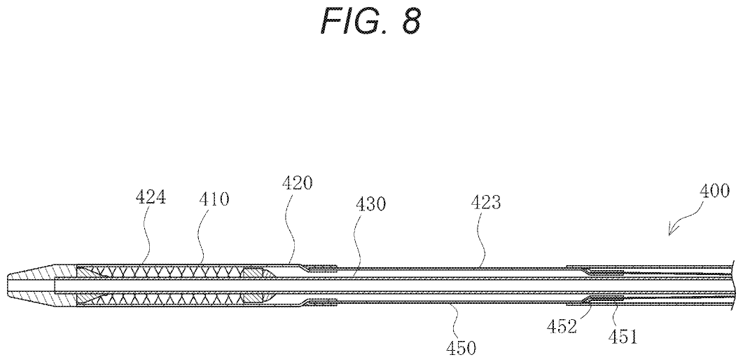

As illustrated in FIG. 7A, a core member 330 is inserted into the formed tube 300. The core member 330 has an outer diameter substantially equal to the inner diameter of the second region 152 of the first sheath 34 to be manufactured. The outer diameter of the core member 330 is 1.3 mm to 1.5 mm.

Next, the tube 300 is inserted into a mold 320. As illustrated in FIG. 7(b), the mold 320 includes a second region forming portion 321 having an inner diameter smaller than the outer diameter of the tube 300, a first region forming portion 322 having an inner diameter substantially the same as the outer diameter of the tube 300, and an intermediate portion 323 inclined such that the diameter decreases from the first region forming portion 322 toward the second region forming portion 321. The inner diameter of the second region forming portion 321 of the mold 320 is 1.6 mm to 1.8 mm. The mold 320 can be heated, and the tube 300 can be heated to a high temperature by heat from the mold 320 to perform diameter reduction processing.

When one side of the tube 300 is inserted into the mold 320 along the longitudinal axis direction and pulled toward the proximal end, the intermediate portion 323 and the second region forming portion 321 reduce the outer diameter of the tube 300, stretch the reinforcing body 312 in the longitudinal axis direction, and increase the pitch of the repeating structure 313. As a result, a part to be the first region 151, a part to be the second region 152, and a part to be the first tapered portion 153 can be formed in the tube 300. The reinforcing body 312 included in the tube 300 is formed of the strands 314 continuous from the distal end to the proximal end of the tube 300, and the tube 300 including the reinforcing body 312 is subjected to diameter reduction processing to form the sheath 33. For this reason, the formed sheath 33 has the reinforcing body 157 formed of the strands 314 continuous from the distal end to the proximal end.

In the first sheath 34 formed in this manner, in the second region 152 positioned at the proximal end of the first region 151, the pitch P2 of the reinforcing body 157 is larger than the pitch P1 in the first region 151, and the inclination angle α2 of the strand 158 with respect to the longitudinal axis direction is smaller than the inclination angle α1 in the first region 151. In the first region 151, the sheath 33 is pulled by the traction wire 80, and accordingly, the strand 158 moves, fixation of the resin existing around the strand 158 is loosened, and the resin is easily peeled off. On the other hand, in the second region 152, since the strand 158 is already pulled in the longitudinal axis direction at the time of manufacturing, even when the sheath 33 is pulled by the traction wire 80, the fixation of the resin existing around the strand 158 is not loosened, and the resin is hardly peeled off. Therefore, in the first sheath 34, the tensile strength in the longitudinal axis direction can be made higher in the second region 152 than in the first region 151.

Here, the manufacturing process of the first sheath 34 has been described, but the second sheath 35 can also be manufactured in a similar manner. The core member used in manufacturing the second sheath 35 has an outer diameter of 1.2 mm to 1.4 mm. The mold used for manufacturing the second sheath 35 includes a third region forming portion having an inner diameter substantially the same as the outer diameter of the third region 161 and a fourth region forming portion having an inner diameter substantially the same as the outer diameter of the fourth region 162, and the inner diameter of the fourth region forming portion is 1.2 mm to 1.4 mm. The first sheath 34 and the second sheath 35 can be connected by covering the first sheath 34 and the second sheath 35 with the connection tube 60. In addition, the traction wire 80 can be fixed by covering the fourth region 162 of the second sheath 35 with the wire fixing tube 65.

Next, a catheter 400 of a first modified example will be described. As illustrated in FIG. 8, the catheter 400 includes a stent 410, an outer tube 420 accommodating the stent 410, and an inner tube 430 disposed inside the outer tube 420, and the basic structure is similar to that of the catheter 10 described above. Description of a part having the same structure as the catheter 10 will be omitted.

The outer tube 420 has a stent sheath 424 connected to the distal side of the sheath 423 and accommodating the stent 410. The traction wire 440 is fixed to the proximal portion of the sheath 423. The sheath 423 includes a second region 451 having a smaller diameter than the first region 450 on the proximal side of the first region 450 on the distal side, and a first tapered portion 452 is formed between the first region 450 and the second region 451. The sheath 423 includes a reinforcing body obtained by braiding metal strands in a resin tube body over the entire length from the distal end to the proximal end. In the reinforcing body, a pitch in the second region 451 is larger than a pitch in the first region 450. The stent sheath 424 is a resin tube body having no reinforcing body. In this manner, the stent sheath 424 accommodating the stent 410 on the distal side may not have the reinforcing body.

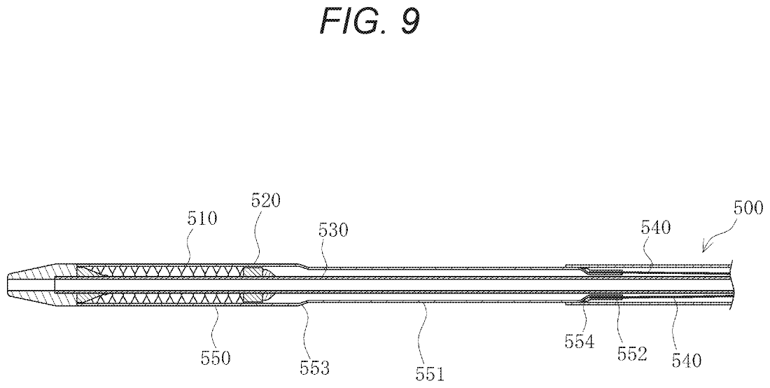

Next, a catheter 500 of a second modified example will be described. As illustrated in FIG. 9, the catheter 500 includes a stent 510, an outer tube 520 accommodating the stent 510, and an inner tube 530 disposed inside the outer tube 520, and the basic structure is similar to that of the catheter 10 described above. Description of a part having the same structure as the catheter 10 will be omitted.

The outer tube 520 has a sheath 523. The sheath 523 has a first region 550 on the distal side, a second region 551 having a smaller diameter than the first region 550 on the proximal side of the first region 550, and a fifth region 553 having a smaller diameter than the second region 551 on the proximal side of the second region 551. The sheath 523 has a first tapered portion 553 between the first region 550 and the second region 551, and a second tapered portion 554 between the second region 551 and the fifth region 553. The traction wire 540 is fixed to the fifth region 552 which is the proximal portion of the sheath 523.

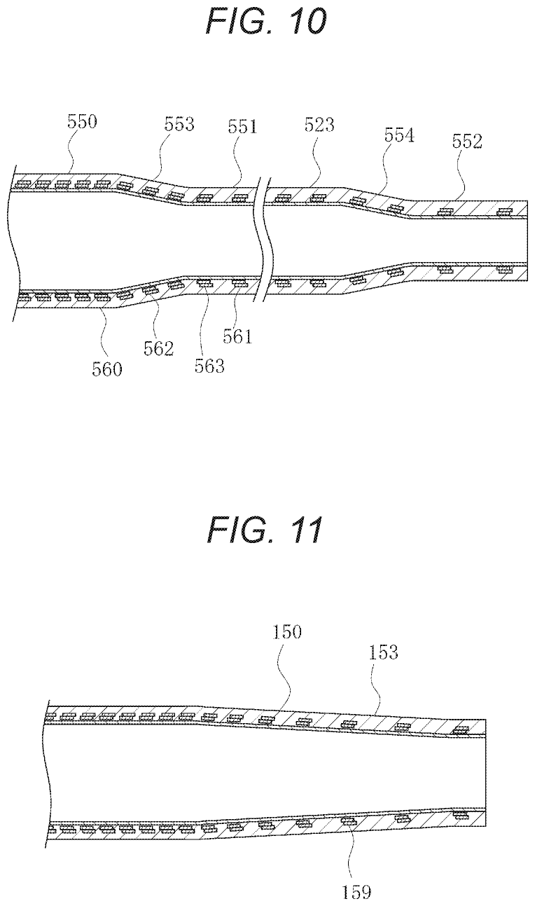

As illustrated in FIG. 10, the sheath 523 includes a reinforcing body 561 having a repeating structure 563 in which metal strands 562 are braided in a resin tube body 560 over the entire length from the distal end to the proximal end. The pitch of the repeating structure 563 of the reinforcing body 561 is larger in the first tapered portion 553 than in the first region 550, larger in the second region 551 than in the first tapered portion 553, larger in the second tapered portion 554 than in the second region 551, and larger in the fifth region 553 than in the second tapered portion 554. That is, as the diameter of the tube body 560 decreases, the pitch of the repeating structure 563 increases.

Next, a modified example of the first sheath 150 will be described. As illustrated in FIG. 11, in the first sheath 150, the first tapered portion 153 may be formed long along the longitudinal axis direction, and the diameter of the first sheath tube body 150 may gradually decrease toward the proximal side. The first sheath reinforcing body 157 is formed such that the pitch of the repeating structure 159 gradually increases in the first tapered portion 153.

As described above, the catheter 10 according to the present embodiment is the catheter 10 including the tubular sheath 33 having the distal end and the proximal end, in which the sheath 33 includes the tube body 150 formed of resin and the reinforcing body 157 having the repeating structure 159 formed of the metal strands 158, the tube body 150 and the reinforcing body 157 continuously extend over the entire length from the distal end to the proximal end of the sheath 33, the sheath 33 has the traction wire 80 fixed to the proximal portion, and the sheath 33 is movable toward the proximal side by pulling the traction wire 80. The catheter 10 configured as described above has the reinforcing body 157 extending over the entire length of the sheath 33 that is pulled by the traction wire 80, and thus the sheath 33 has continuous tensile strength in the longitudinal axis direction from the distal end to the proximal end, and a local decrease in tensile strength can be prevented from occurring.

In the catheter 10 described above, the sheath 33 may include the first region 151 and the second region 152 at the proximal end of the first region 151, and the second region 152 may have a smaller outer diameter than the first region 151 and a larger pitch of the repeating structure 159 forming the reinforcing body 157. As a result, the catheter 10 can have high tensile strength in the longitudinal axis direction in the second region 152.

In the catheter 10 described above, the sheath 33 may include the first region 151 and the second region 152 at the proximal end of the first region 151, and the second region 152 may have an outer diameter smaller than that of the first region 151, and a smaller angle formed by the strands 158 in the repeating structure 159 forming the reinforcing body 157 with respect to the longitudinal axis direction of the sheath 33. As a result, the catheter 10 can have high tensile strength in the longitudinal axis direction in the second region 152.

In the catheter 10 described above, the sheath 33 may include the tapered portion 153 between the first region 151 and the second region 152, the tapered portion being inclined such that the diameter decreases from the first region 151 toward the second region 152, and the pitch of the repeating structure 159 in the tapered portion 153 may be greater than the pitch of the repeating structure 159 in the first region 151 and smaller than the pitch of the repeating structure 159 in the second region 152. As a result, the catheter 10 can continuously change the tensile strength of the sheath 33 between the first region 151 and the second region 152, and suppress breakage by suppressing abrupt physical property changes of the sheath 33.

In the catheter 10 described above, the first region 151 of the sheath 33 may be a region that accommodates the stent 20 at least in part. As a result, the catheter 10 can accommodate the stent 20 in a state of reliably holding the stent 20 inside the first region 151 of the sheath 33.

In the catheter 10 described above, the sheath 33 may include the first sheath 34 that accommodates the stent 20, and the second sheath 35 connected to the proximal side of the first sheath 34 and including the resin tube body 150 and the reinforcing body 167 formed by braiding metal strands 158. As a result, when the catheter 10 has the first sheath 34 that accommodates the stent 20 and the second sheath 35 that is accommodated in a shaft on the proximal side as separate components, the catheter 10 can continuously change the tensile strength along the longitudinal axis direction over the entire sheath 33, and prevent a local decrease in tensile strength from occurring.

In the catheter 10 described above, the traction wire 80 may be fixed to the proximal portion of the second sheath 35. As a result, the catheter 10 can smoothly pull the second sheath 35.

In the catheter 10 described above, the second sheath 35 may include the third region 161 and the fourth region 162 at the proximal end of the third region 161, and the fourth region 162 may have a smaller outer diameter than the third region 161 and a larger pitch of the repeating structure 169 forming the reinforcing body 167. As a result, the catheter 10 can increase the tensile strength in the longitudinal axis direction in the fourth region 162 of the second sheath 35.

In the catheter 500 described above, the sheath 523 may include the fifth region 553 at the proximal end of the second region 551, and the fifth region 553 may have a smaller outer diameter than the second region 551 and a larger pitch of the repeating structure 563 forming the reinforcing body 561. As a result, when the catheter 500 has a part that accommodates the stent 510 and a part that is accommodated in a shaft on the proximal side integrally, the catheter 500 can continuously change the tensile strength along the longitudinal axis direction over the entire sheath 523, and prevent a local decrease in tensile strength from occurring.

In the catheter 400 described above, the stent sheath 424 that accommodates the stent 410 may be connected to the distal side of the sheath 423, and the stent sheath 424 may be a resin tube body that does not have the reinforcing body. As a result, the catheter 400 can increase the flexibility of the part that accommodates the stent 410.

In the catheter 10 described above, the traction wire 80 may be fixed to the fixing region of the sheath 33, and the fixing region may have the reinforcing body 167 extending at least in part. As a result, the catheter 10 can increase the tensile strength in the fixing region of the sheath 33 that is pulled by the traction wire 80 and can suppress breakage of the sheath 33.

The method for manufacturing the catheter 10 according to the present embodiment is a method for manufacturing the catheter 10 provided with the tubular sheath 33 having a distal end and a proximal end, the method including: a step of forming a tube including a reinforcing body having the repeating structure 159 formed of metal over the entire length of the resin tube body 150; and a step of inserting one side of the tube along the longitudinal axis direction up to an intermediate position into a mold having an inner diameter smaller than the outer diameter of the tube, and increasing the pitch of the repeating structure 159 while reducing the outer diameter of the tube that has passed through the mold. In the method for manufacturing the catheter 10 configured as described above, since the sheath 33 is formed by performing diameter reduction processing on the tube including the reinforcing body continuous from the distal end to the proximal end of the tube, the formed sheath 33 has the reinforcing body continuous from the distal end to the proximal end, the sheath 33 has continuous tensile strength from the distal end to the proximal end in the longitudinal axis direction, and a local decrease in tensile strength can be prevented from occurring.

Note that the present invention is not limited to the embodiment described above, and those skilled in the art can make various modifications within the technical scope of the present disclosure. In the above-described embodiment, the catheter in which the stent is placed has been described, but the present invention can also be applied to other types of catheters as long as the catheter includes a tubular sheath, and for example, can also be applied to an ultrasonic catheter or the like.

In the sheath 33, in the second region 152 positioned at the proximal end of the first region 151, the pitch P2 of the reinforcing body 157 is larger than the pitch P1 in the first region 151, and the inclination angle α2 of the strands 158 with respect to the longitudinal axis direction is smaller than the inclination angle α1 in the first region 151. However, in the second region 152, the pitch P2 of the reinforcing body 157 may be smaller than the pitch P1 in the first region 151, and the inclination angle α2 of the strands 158 with respect to the longitudinal axis direction may be larger than the inclination angle α1 in the first region 151.

What is claimed is:

1. A catheter comprising:
a tube;
a sheath surrounding the tube and including:
a tube body formed of resin, and
a reinforcing body having a repeating structure formed of metal strands; and
a traction wire connected to a proximal portion of the sheath and configured to move the sheath with respect to the tube, wherein
the tube body and the reinforcing body extend continuously over an entire length of the sheath.

2. The catheter according to claim 1, wherein
the sheath includes a first region and a second region that is closer to a proximal end of the catheter than the first region,
an outer diameter of the second region is smaller than an outer diameter of the first region, and
a pitch of the repeating structure of the reinforcing body in the second region is greater than a pitch of the repeating structure in the first region.

3. The catheter according to claim 2, wherein
the sheath includes a tapered portion between the first and second regions, an outer diameter of the tapered portion decreasing from the first region toward the second region, and
a pitch of the repeating structure of the reinforcing body in the tapered portion is greater than the pitch of the repeating structure in the first region and less than the pitch of the repeating structure in the second region.

4. The catheter according to claim 2, further comprising:
a stent that is at least partially accommodated in the first region.

5. The catheter according to claim 4, wherein
the sheath includes a first sheath that accommodates the stent and a second sheath connected to a proximal side of the first sheath, the second sheath including a second tube body formed of resin and a second reinforcing body having a repeating structure formed of metal strands.

6. The catheter according to claim 5, wherein the traction wire is fixed to a proximal portion of the second sheath.

7. The catheter according to claim 5, wherein
the second sheath includes a third region and a fourth region that is closer to the proximal end of the catheter than the third region,
an outer diameter of the fourth region is smaller than an outer diameter of the third region, and
a pitch of the repeating structure of the second reinforcing body in the fourth region is greater than a pitch of the repeating structure in the third region.

8. The catheter according to claim 2, wherein
the sheath includes a fifth region at a proximal end of the second region,
an outer diameter of the fifth region is smaller than the outer diameter of the second region, and
a pitch of the repeating structure of the reinforcing body in the fifth region is greater than the pitch of the repeating structure in the second region.

9. The catheter according to claim 2, further comprising:
a stent sheath that accommodates a stent and is connected to a distal side of the sheath, wherein
the stent sheath includes a tube body formed of resin.

10. The catheter according to claim 9, wherein the stent sheath does not include a reinforcing body.

11. The catheter according to claim 1, wherein
the sheath includes a first region and a second region that is closer to a proximal end of the catheter than the first region,
an outer diameter of the second region is smaller than an outer diameter of the first region, and
when viewed from a direction perpendicular to an axis direction of the sheath, an angle formed by each of the metal strands of the reinforcing body in the second region with respect to the axis direction is smaller than an angle formed by each of the metal strands of the reinforcing body in the first region with respect to the axis direction.

12. The catheter according to claim 1, wherein the tube is an inner tube including a guide wire lumen.

13. The catheter according to claim 12, wherein
the inner tube includes a distal tip fixed to a distal portion of the inner tube, and
the guide wire lumen opens at a distal end of the distal tip.

14. The catheter according to claim 1, wherein
the tube body of the sheath includes an outer layer formed of a flexible resin and an inner layer formed of a low-friction resin, and
the reinforcing body is between the outer and inner layers.

15. The catheter according to claim 1, further comprising:
a proximal outer tube positioned on a proximal side of the sheath, wherein
the sheath is slidable on an inner surface of the proximal outer tube and enters an inside of the proximal outer tube when the sheath moves toward the proximal side.

16. The catheter according to claim 1, wherein
the sheath includes a first sheath and a second sheath,
a distal portion of the second sheath is positioned on an outer surface of a proximal portion of the first sheath, and
the first and second sheaths are connected by a connection tube covering the distal portion of the second sheath and the outer surface of the proximal portion of the first sheath.

17. The catheter according to claim 1, wherein the traction wire is positioned on an outer surface of the proximal portion of the sheath and is fixed to the sheath by a wire fixing tube covering the traction wire.

18. The catheter according to claim 1, wherein the catheter is a rapid exchange type catheter.

19. A method for manufacturing a catheter that includes a sheath, the method comprising:
forming a tube that includes a tube body formed of resin and a reinforcing body having a repeating structure formed of metal strands, the tube body and the reinforcing body extending continuously over an entire length of the tube; and
inserting one side of the tube along an axis direction up to an intermediate position into a mold having an inner diameter smaller than an outer diameter of the tube, and increasing a pitch of the repeating structure while reducing the outer diameter of a portion of the tube that has passed through the mold to form the sheath.

20. The method according to claim 19, further comprising:
inserting a core member into the tube prior to inserting the tube into the mold, wherein
the mold is heated to reduce the outer diameter of the portion of the tube.
